(19) United States
(12) Patent Application Publication
(10) Pub. No.: US 2026/0283208 A1
(43) Pub. Date: Sep. 24, 2026

(54) HUMAN MILK OLIGOSACCHARIDES

(71) Applicant: Chr. Hansen A/S, Hoersholm (DK)

(72) Inventors: Stina JENSEN, Hoersholm (DK); Daniel VILLALBA, Hoersholm (DK)

(73) Assignee: Chr. Hansen A/S, Hoersholm (DK)

(21) Appl. No.: 19/476,108

(22) PCT Filed: Apr. 18, 2024
(86) PCT No.: PCT/EP2024/060503

§ 371 Date: Oct. 16, 2025

(30) Foreign Application Priority Data
Apr. 19, 2023 (EP) 23168747.6

(51) Int. Cl. A23L 33/125; A23L 33/00; A23L 33/135

(52) U.S. Cl. CPC A23L 33/125; A23L 33/135; A23L 33/40

(57) ABSTRACT

The present disclosure relates a composition for use in balancing the immune system of a newborn or infant subject, said composition comprising an effective amount of 2′-fucosyllactose, 3-fucosyllactose, 3′-sialyllactose, 6′-sialyllactose, and lacto-N-tetraose.

### FIELD

The present disclosure relates, in part, to the use of a mix of human milk oligosaccharides for modulating immune responses in a subject. Further disclosed herein are methods, uses, processes, and the like.

### BACKGROUND

Human milk oligosaccharides (HMOs) are non-digestible carbohydrates found in human milk. Their importance to infant nutrition is underscored by their position as the third most abundant solid component of human milk, behind lactose and lipids. HMOs can be structurally categorized as (a) fucosylated HMOs such as 2′- and 3-fucosyllactose (2′-FL and 3-FL), (b) neutral non-fucosylated HMOs such as lacto-N-tetraose (LNT) and (c) sialylated HMOs such as 3′- and 6′ sialyllactose (3′-SL and 6′-SL).

HMOs in human milk vary widely based on various influences such as genetics, lactation, and geographic location. While most HMO concentrations decrease over the course of lactation, at least two, 3′-SL and 3-FL, may increase. Different HMOs may work together in complementary ways to support the growth and development of infants. HMOs are thought to have various biological functions, such as preventing the attachment of pathogens to epithelial cells (Ruiz-Palacios, Cervantes, Ramos, Chavez-Munguia, & Newburg, 2003), modulating immune cell responses (Zhang et al., 2019) in vitro, modulating gut microbiota (Berger et al., 2020; Elison et al., 2016; Iribarren et al., 2020). Specific stimulation of gut bacteria by fucosylated HMOs (2′FL, 3FL and DFL) has been seen in an in vitro anaerobic culture system using infant fecal microbiota (Yu et al., 2013). It has been suggested that HMOs can lower the risk of gut microbiome imbalance due to harmful bacteria (Weichert, Stefan, et al. Nutrition research10 (2013): 831-838). Further, it has been suggested that HMOs can selectively stimulate helpful bifidobacteria in support of overall gut health (Bode, Lars. Nutrition reviews suppl_2 (2009)), support microbial colonization and gut barrier function (Kong et. al., Mol. Nutr. Food Res. 2019, 63), and enhance mucus barrier function through direct modulation of intestinal goblet cells (Cheng et. al., Mol. Nutr. Food Res. 2020, 64).

Infants display a heightened susceptibility to suffer from infections and allergic diseases as they display a skewed T_{H}2 and impaired T_{H}1-immunity (Debock et al. Unbalanced neonatal CD4+ T-cell immunity-Immunol., 27 Aug. 2014 Vol. 5 (2014)). It is believed the infant immune system is biased toward a TH2 response as this supports a more tolerogenic phenotype, which is important both during pregnancy and in early life. However, excessive T_{H}2 immune response can lead to adverse conditions such as allergic responses. As the infant matures, other immune response such as T_{H}1 support full immune protection against viruses and bacteria. Therefore, it is desirable to aid in the development of a balanced immune system. That is, an immune system that is less biased toward a T_{H}2 response. Different cytokines are indicative of different types of immune response. For example, IFN-γ and IL-2 are associated with T_{H}1 responses whereas IL-4, IL-5 and IL-13 are associated with T_{H}2 responses.

Early life vaccination strategies aim to compensate this to support and mature the immune system of infants. Nevertheless, vaccines such as DTaP provide ineffective long-lasting humoral and cell-mediated immune protection against Bortedella pertussis (PT). Breastfeeding is seen to improve response to vaccines in the still maturing immune system of infants. HMOs have been shown to mature the immune system of infants and directly modulate the function of immune cells including DCs, MØ and T cells. Nevertheless, the direct effects of HMOs on innate and adaptive immune responses remains unclear.

While the effects of individual HMOs have been extensively investigated, there has been less research into combinations.

### SUMMARY

The present disclosure provides compositions, uses, methods and the like for balancing the immune system of a subject. In particular, the present disclosure relates to compositions comprising 5 HMOS (2′-Fucosyllactose (2′-FL), 3-Fucosyllactose (3-FL), 3′-sialyllactose (3′-SL), 6′-sialyllactose (6′-SL) and lacto-N-tetraose (LNT)) for balancing the immune system of non-adult humans.

The present disclosure provides compositions for the modulation of immune activity such as allergies, and the like.

While not wishing to be bound by theory, it is believed the 5 HMO mix balances the amount of cytokines that promote T_{H}1-related responses and the amount of cytokines that promote T_{H}2-related responses. This balance helps avoid any one type of response predominating and allows for the immune system to respond appropriately to challenges. Moreover, the 5 HMO mix also appears to enhance cytokines related to the T_{H}17 immune response, which could be important for development of the immune system.

### BRIEF DESCRIPTION OF THE DRAWINGS

FIG. 1 shows the effect of 5 HMO mix on enhancing release of Th1 priming interleukin (IL)-12 from LPS-activated human dendritic cells (DC) and macrophages (MØ). In the presence of IL-12, naïve CD4+ T cells differentiate towards Th1 rather than the Th2 direction. Th1 cells produce large amounts of interferon (IFN)-γ.

FIG. 2 shows the effect of 5 HMO mix on enhancing release of the Th2 priming IL-4 from LPS-activated human dendritic cells (DC) and macrophages (MØ). In the presence of IL-4, naïve CD4+ T cells favor differentiation into Th2 rather than the Th1 direction. Th2 cells produce large amounts of IL-4, IL-5, and IL-13.

FIG. 3 shows the effect of 5 HMO mix on the Th2/Th1 ratio. Specifically, DCs or MØ activated with LPS in the presence or absence of 5 HMO mix were co-cultured with naïve CD4 T cells, after which the amounts of Th1 signature cytokine IFN-γ (see also FIG. 5) and Th2 signature IL-13 (see also FIG. 6) released was quantified and used to calculate the IL-13/IFN-γ ratio.

FIG. 4 shows the classification of macrophages into: 1) pro-inflammatory M1 macrophages, which are pro-inflammatory by nature and critical for host protection against viruses and intracellular bacteria; and 2) M2 macrophages, which are anti-inflammatory by nature and play an important role in tissue repair. The macrophage experiments described herein are all performed using M1 macrophages.

FIG. 5 shows the effect of 5 HMO mix on IFN-y release in a co-culture system of human LPS-activated DCs and naïve CD4+ T cells or LPS-activated MØ and naïve CD4+ T cells. The 5 HMO mix significantly boosts IFN-γ release in a dose dependent manner in both co-culture systems compared to the respective LPS only groups.

FIG. 6 shows the effect of 5 HMO mix on IL-13 release in a co-culture system of human LPS-activated DCs and naïve CD4+ T cells or LPS-activated MØ and T-lymphocytes. In both co-culture systems the 5 HMO mix significantly decreases IL-13 release compared to the respective LPS only groups, which can be an effector of allergic responses if dominating.

FIG. 7 shows the effect of 5 HMO mix on release of T_{H}17 priming cytokines IL-6 and IL-23 from LPS-activated DCs, as well as on release of T_{H}17 signature effector cytokine IL-17 from naïve CD4+ T cells co-cultured with LPS-activated DCs. The 5 HMO mix significantly increases DC release of IL23 and IL-6 as well as CD4 T cell release of IL-17 in a dose dependent manner.

FIG. 8 shows the effect of 5 HMO mix on release of the anti-inflammatory cytokine IL-10 from LPS-activated DCs and MØs. 5 HMO mix significantly increases IL-10 secretion from DCs and MØ compared to the respective LPS only groups, and thereby ensures a regulatory environment that adds to a balanced immune system.

FIG. 9 shows the antibody response to Bordetella pertussis toxin (PT) in serum of mice administered a Diphtheria-Tetanus-Acellular Pertussis (DTaP) vaccine on Days 0, 14 and 28 and receiving oral gavage of 5 HMO mix at different dosages (0.01, 0.05 and 0.1% of bodyweight) starting 14 days prior to initial vaccination. 5 HMO mix supplementation enhances serum levels of PT-specific IgG on days 28 and 42/43 post initial vaccination. Statistical significance was determined by a Kruskal-Wallis non-parametric test followed Dunn's multiple comparisons test. **, p<0.01; *p<0.05 indicates significant differences from the DTaP only group at respective time points.

FIG. 10 shows the cell-mediated immune response to PT of splenocytes isolated on the day of euthanization (day 42 or 43) from the mouse included in the DTaP vaccine study described in FIG. 8. 5 HMO mix supplementation enhances the frequency of T-bet-expressing CD4+ T helper cells (Th1 cells) and tumor necrosis factor (TNF)-a expressing CD8+ cytotoxic T cells in splenocytes of DTaP-vaccinated mice stimulated ex vivo with 8 μg/mL PT. Stimulation with 12-myristate 13-acetate/ionomycin (PMA-IONO) was included as a positive control. Statistical significance was determined by a Kruskal-Wallis non-parametric test followed Dunn's multiple comparisons test. ***, p<0.001; **, p<0.01; *p<0.05 indicates significant differences from the DTaP only group.

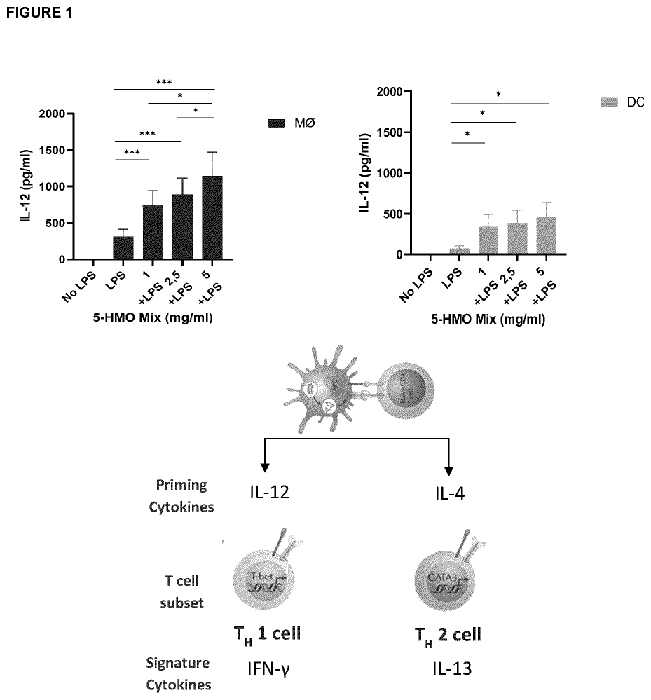

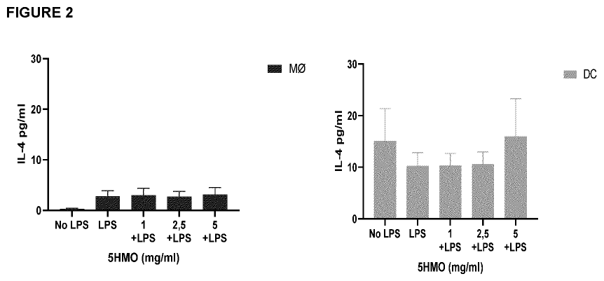

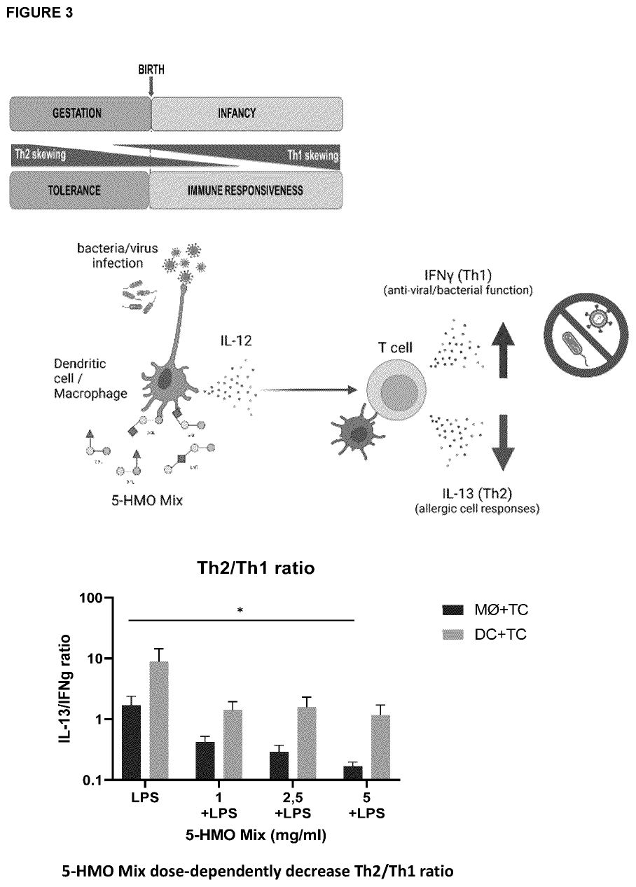

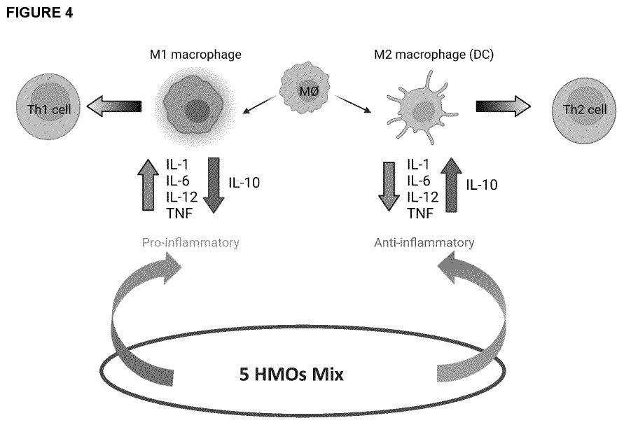

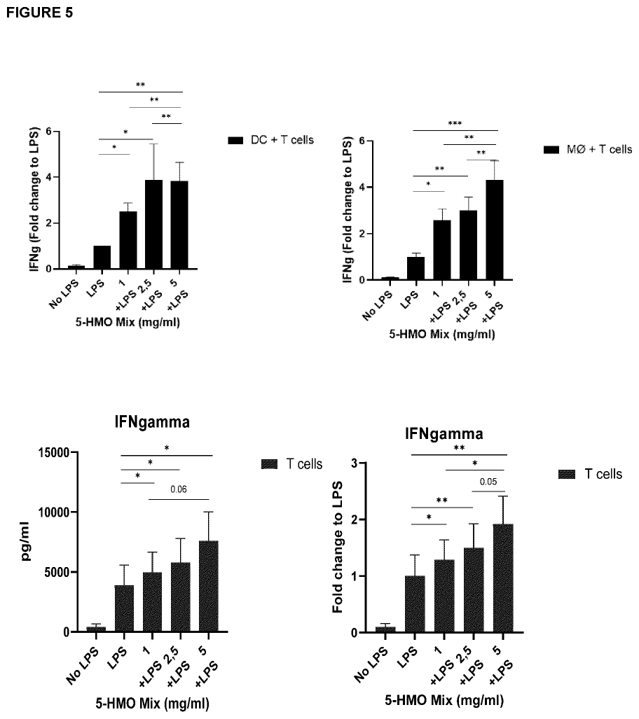

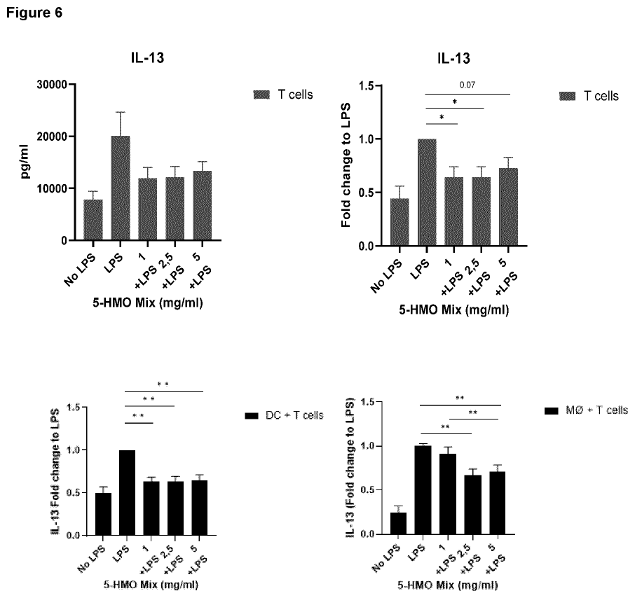

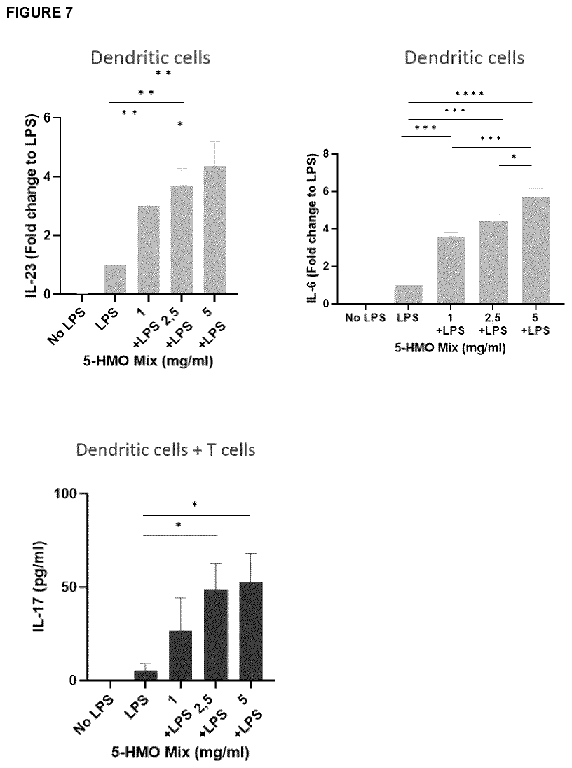

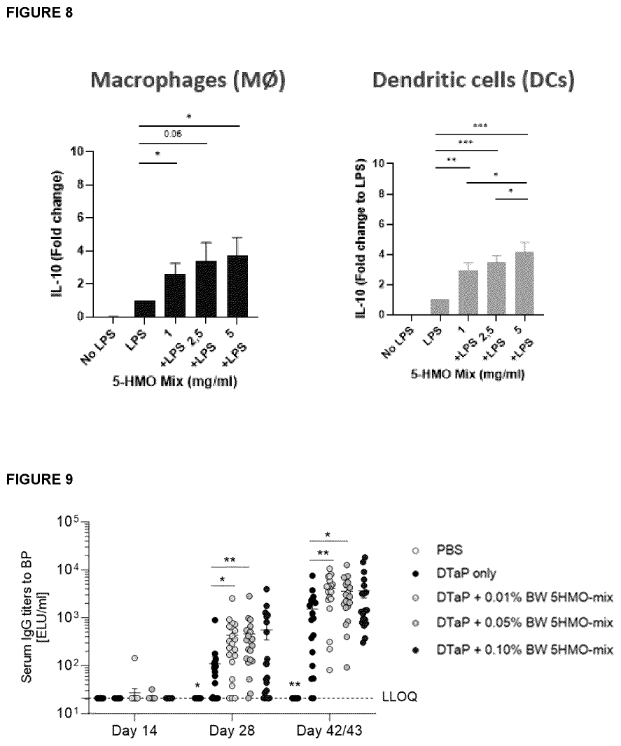

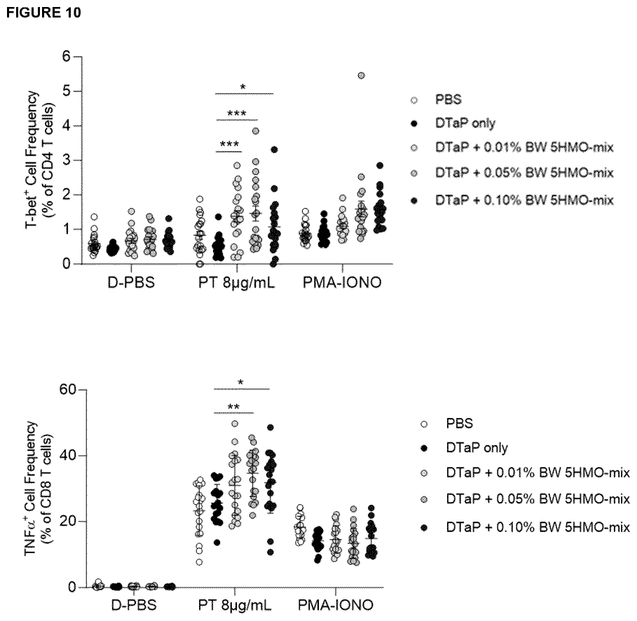

### DETAILED DISCRIPTION

Unless defined otherwise, all technical and scientific terms used herein have the same meaning as commonly understood by persons skilled in the art. Although any methods and materials equivalent or similar to those described herein can be used in the practice of the present disclosure, typical methods and materials are described. All methods described herein can be performed in any suitable order unless otherwise indicated herein or otherwise clearly contradicted by context.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range including the two end values, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed.

As used herein, the term “and/or” is intended to mean the combined (“and”) and the exclusive (“or”) use, i.e. “A and/or B” is intended to mean “A alone, or B alone, or A and B together”.

As used herein the terms “effective amount”, “effective concentration”, or “effective dosage” are defined as the amount, concentration, or dosage of a material sufficient to improve the overall health of the animal and confer benefits similar to the ones demonstrated in the examples. The actual effective dosage in absolute numbers depends on factors including the state of health of the subject in question, and other ingredients present. The “effective amount”, “effective concentration”, or “effective dosage” of the material may be determined by routine assays known to those skilled in the art.

As used herein the term “isolated” means that the bacterial strains described herein are in a form or environment which does not occur in nature, i.e. the strain is at least partially removed from one or more or all of the naturally occurring constituents with which it is associated in nature.

A bacterial “strain” as used herein refers to a bacterium which remains genetically unchanged when grown or multiplied and that originates from a single isolate or pure culture. Probiotics are classified by their genus (e.g. Bifidobacterium), species and subspecies (e.g. animalis subsp. lactis), and strains (e.g. DSM 15954 and/or BB-12®). FAO/WHO has stated that probiotic effects are strain specific and that most probiotic characteristics of a particular strain cannot therefore be extrapolated to other strains of the same species.

As used herein, the term “probiotic” refers to a culture of live or freeze-dried microorganisms, dead microorganisms, fragments of microorganisms and extracts or supernatants of microorganisms which, when applied to man or animal, beneficially affects the host (Hill et al. (2014) Expert Consensus Document, The International Scientific Association for Probiotics and Prebiotics. Consensus statement on the scope and appropriate use of the term probiotic).

The term “human milk oligosaccharide” or “HMO”, as used herein, unless otherwise specified, refers generally to a number of complex carbohydrates found in human breast milk that can be in acidic or neutral form, and to precursors thereof. Exemplary non-limiting human milk oligosaccharides include 3′-sialyllactose, 6′-sialyllactose, 3-fucosyllactose, 2′-fucosyllactose, and lacto-N-neo-tetraose.

The terms “treat” or “treating” should not be taken to imply that an individual is treated until total recovery. Accordingly, these terms broadly include amelioration and/or prevention of the onset of the symptoms or severity of a particular condition.

The term “shelf stable” as used herein, unless otherwise specified, refers to a nutritional product that remains commercially stable after being packaged and then stored at 18-24° C. for at least 3 months, including from about 6 months to about 24 months, and also including from about 12 months to about 18 months.

The terms “nutritional formulation” or “nutritional composition” as used herein, are used interchangeably and, unless otherwise specified, refer to nutritional liquids, nutritional powders, nutritional supplements, and any other nutritional food product as known in the art. The nutritional powders may be reconstituted to form a nutritional liquid, all of which comprise one or more of fat, protein and carbohydrate and are suitable for oral consumption by a human.

The term “nutritional powder” as used herein, unless otherwise specified, refers to nutritional products in flowable or scoopable form that can be reconstituted with water or another aqueous liquid prior to consumption and includes both spray-dried and dry-mixed dry-blended powders.

The term “newborn” as used herein, unless otherwise specified, refers to a person from birth up to four weeks of age. The term “infant” as used herein, unless otherwise specified, refers to a person 12 months or younger. The term “preterm” as used herein, refers to a baby born prior to 36 weeks of gestation. The term “toddler” as used herein, unless otherwise specified, refers to a person greater than one year of age up to three years of age. The term “child” as used herein, unless otherwise specified, refers to a person greater than three years of age up to twelve years of age.

The term “formula” as used herein, unless otherwise specified, refers to liquid and solid human milk replacements or substitutes that are suitable for consumption by a human.

The term “human milk fortifier” as used herein, unless otherwise specified, refers to liquid and solid nutritional products suitable for mixing with breast milk or formula for consumption by a preterm or term infant.

The terms “susceptible” and “at risk” as used herein, unless otherwise specified, mean having little resistance to a certain condition or disease, including being genetically predisposed, having a family history of, and/or having symptoms of the condition or disease. The terms “modulating” or “modulation” or “modulate” as used herein, unless otherwise specified, refer to the targeted movement of a selected characteristic.

All percentages, parts and ratios as used herein, are by weight of the total composition, unless otherwise specified. All such weights, as they pertain to listed ingredients, are based on the active level and, therefore, do not include solvents or by-products that may be included in commercially available materials, unless otherwise specified.

Numerical ranges as used herein are intended to include every number and subset of numbers within that range, whether specifically disclosed or not.

The present invention has been described with reference to various embodiments, aspects, examples, or the like. It is not intended that these elements be read in isolation from one another. Thus, the present disclosure provides for the combination of two or more of the embodiments, aspects, examples, or the like.

All embodiments described herein are intended to be within the scope of the invention disclosed. These and other embodiments of the present invention will become readily apparent to those skilled in the art from the following detailed description of the preferred embodiments having reference to the whole description, the invention not being limited to any particular preferred embodiment(s) disclosed. No language in the specification should be construed as indicating any non-claimed element as essential to the practice of the invention.

The present disclosure provides compositions comprising a mixture of 5 HMOS (2-FL, 3-FL, 3-SL, 6-SL and LNT) that promotes balancing the immune system. As used herein, the phrase ‘balancing the immune system’ refers to the ratio of Th1 and Th2 related cytokines. While not wishing to be bound by theory, it is believed that newborns and infants with a more balanced immune system will be less susceptible to pathogens and show less propensity to develop allergic diseases. Modulation of the immune system is also supported by the 5 HMO mix effect on mediating a T_{H}17 immune response that supports protection to extracellular pathogens and fungi.

In certain embodiments, the present disclosure provides compositions comprising a mixture of 5 HMOs for modulating the secretion of pro-inflammatory cytokines (e.g. IL-12, IL-6, IL-23) and anti-inflammatory cytokines (e.g. IL-10 from human MØ and DCs) such that the ratio is more balanced. When challenged with LPS the 5 HMO mix stimulated MØ and DCs enhanced T_{H}1-(IFN-γ production) whilst down-regulating T_{H}2 cell responses (lower IL-13).

In vitro and in vivo immunological effects of 5 HMO mix on innate and adaptive immunity were examined. The direct effect of a mixture of 5 HMOS (2-FL, 3-FL, 3-SL, 6-SL, LNT) in different immune cell types including DCs, MØ, CD4⁺ and CD8⁺ T cells was tested. The 5 HMO mix as a dietary intervention that could enhance immune responsiveness against ertussis in a specific DTaP vaccination mice model was also examined. It was found that the 5 HMO mix dose-dependently induces the production of IL-12, IFNy, IL6, IL-23, IL-17 and IL-10, but not IL-4 and IL-13 in LPS-challenged human DCs and MØ. Consistently, LPS+5 HMO mix-stimulated MØ and DCs significantly induced dose-dependently IFN-γ production from human T cells. LPS+5HMO Mix-primed DCs led to a significant down-regulation in IL-13 production. Thus, 5 HMO mix supplementation balanced the T_{H}1/T_{H}2 ratio immunity and support the maturation of other immune phenotypes including T_{H}17 (IL-6, IL-23 and IL-17). 5-HMO mix supplementation was tested to see if it could improve humoral and adaptive immune responses against bortedella Pertussis (PT) in a specific DTaP vaccination mice model. It was observed that dietary 5-HMO mix enhanced serum levels of anti-PT IgG. Furthermore, the frequency of T_{H}1 cells and TNF-α⁺ CD8⁺ T cells, after ex vivo restimulation with PT, were significantly increased in splenocytes of mice receiving 5-HMO mix as compared to vaccination only control mice group. As neonates display an immature immune system and susceptibility to suffer from infections, the data suggest that 5 HMO mix supplementation will be favorable to enhance humoral and cell-mediated immunity in infants and support the efficacy of the vaccine against PT.

The present compositions may be used to improve the efficacy of a vaccine. For example, the present compositions may be administered to a subject who has recently received, or is about to receive a vaccine, in order to enhance the immune response to said vaccine. The subject may, for example, be a newborn. The subject may, for example, be an infant. The subject may, for example, be a toddler. The subject may, for example, be a child.

The present compositions comprise five Human Milk Oligosaccharide (HMO) namely, 2′-fucosyllactose, 3-fucosyllactose, 3′-sialyllactose, 6′-sialyllactose, and lacto-N-tetraose. The present compositions may comprise other HMOs such as, for example, lacto-N-neotetraose.

Formulating compositions with HMOs can be somewhat problematic. It has been found that a more reproducible and consistent composition can be achieved through controlling the particle size distribution (PSD) of the HMO. While not wishing to be bound by theory, it is believed that having a somewhat narrow PSD improves the flowability of the HMO enabling a more effective mixing with the other ingredients. In addition, it is believed that a PSD within a certain range provides a better solubility profile. Particle size of an HMO may be determined using a standard method, such as using a sieve tower, which separates the powder into the different fractions after a defined time with a predefined amplitude. The sieves used in such a method may be sieves which comply with DIN ISO 3310-1.

It is preferred that the HMOs used in the present compositions have the following particle size characteristics:
Percent through mesh #230 (63 μm)—less than about 20%, less than about 18%, less than about 16%, less than or equal to about 15%.
Percent through mesh #100 (150 μm)—greater than about 75%, greater than about 70%, greater than about 65%, greater than or equal to about 60%.
Percent through mesh #45 (355 μm)—greater than about 95%, greater than about 92%, greater than or equal to about 90%.
Percent through mesh #20 (850 μm)—100%.

A preferred composition herein is a nutritional composition such as a formula. The nutritional compositions may be in any product form comprising the ingredients described herein, and which is safe and effective for oral administration. The nutritional compositions may be formulated with optional ingredients such as those described herein.

The nutritional compositions of the present disclosure are preferably formulated as dietary product forms, which are defined herein as those embodiments comprising the ingredients of the present disclosure in a product form that then contains at least one of fat, protein, and carbohydrate, and preferably also contains vitamins, minerals, or combinations thereof.

The nutritional compositions may be formulated with sufficient kinds and amounts of nutrients to provide a sole, primary, or supplemental source of nutrition, or to provide a specialized nutritional product for use in individuals afflicted with specific diseases or conditions or with a targeted nutritional benefit as described below. Specific non-limiting examples of product forms suitable for use HMO containing compositions as disclosed herein include, for example, liquid and powdered dietary supplements, liquid and powdered human milk fortifiers, liquid, and powdered formula.

Nutritional liquids include both concentrated and ready-to-feed nutritional liquids. These nutritional liquids are most typically formulated as suspensions or emulsions, although other liquid forms are within the scope of the present disclosure.

Nutritional emulsions suitable for use may be aqueous emulsions comprising proteins, fats, and carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The nutritional emulsions may be and typically are shelf stable. The nutritional emulsions typically contain up to about 95% by weight of water, including from about 50% to about 95%, also including from about 60% to about 90%, and also including from about 70% to about 85%, of water by weight of the nutritional emulsions. The nutritional emulsions may have a variety of product densities, but most typically have a density greater than about 1 g/mL, including greater than about 1.05 g/mL, including greater than about 1.055 g/mL to about 1.12 g/mL, and also including from about 1.085 g/mL to about 1.10 g/mL. The nutritional emulsions may have a caloric density tailored to the nutritional needs of the ultimate user, although in most instances the emulsions comprise generally at least 660 kcal/liter, about 675 kcal/liter to about 820 kcal/liter, about 680 kcal/liter to about 800 kcal/liter. In some embodiments, the emulsion may have a caloric density of from about 50-100 kcal/liter to about 660 kcal/liter, including from about 150 kcal/liter to about 500 kcal/liter. In some specific embodiments, the emulsion may have a caloric density of 25, or 50, or 75, or 100 kcal/liter. The nutritional emulsion may have a pH ranging from about 3.5 to about 8, from about 4.5 to about 7.5, including from about 5.5 to about 7.3, including from about 6.2 to about 7.2. Although the serving size for the nutritional emulsion can vary depending upon a number of variables, a typical serving size is generally at least 1 mL, or even at least 2 mL, or even at least 5 mL, or even at least 10 mL, or even at least 25 mL, including ranges from about 1 mL to about 300 mL, including from about 4 mL to about 250 mL, and including from about 10 mL to about 240 mL.

The nutritional solids may be in any solid form but are typically in the form of flowable or substantially flowable particulate compositions, or at least particulate compositions, that may optionally be compressed into tablets. Particularly suitable nutritional solid product forms include spray dried, agglomerated and/or dry-blended powder compositions. The compositions can easily be scooped and measured with a spoon or similar other device, and can easily be reconstituted by the intended user with a suitable aqueous liquid, typically water, to form a nutritional composition for immediate oral or enteral use. In this context, “immediate” use generally means within about 48 hours, most typically within about 24 hours, preferably right after reconstitution. The nutritional powders may be reconstituted with water prior to use to a caloric density tailored to the nutritional needs of the ultimate user, although in most instances the powders are reconstituted with water to form compositions comprising generally at least 660 kcal/liter, about 675 kcal/liter to about 820 kcal/liter, about 680 kcal/liter to about 800 kcal/liter. In some embodiments, the reconstituted powder may have a caloric density of from about 50-100 kcal/liter to about 660 kcal/liter, including from about 150 kcal/liter to about 500 kcal/liter. In some specific embodiments, the reconstituted powder may have a caloric density of 25, or 50, or 75, or 100 kcal/liter.

The present compositions may be useful in newborns, infants, toddlers, or children. The present compositions may be useful in balancing the immune system in newborns, infants, toddlers, children. The present compositions may be useful in newborns. The present compositions may be useful in infants.

The present compositions may comprise individual HMOs in any suitable amount, such as, for example, at least 0.001 mg/mL, including from about 0.001 mg/mL to about 20 mg/mL, including from about 0.01 mg/mL to about 10 mg/mL, including from about 0.01 mg/mL to about 5 mg/mL (mg of particular HMO per mL of composition).

Where the composition is a nutritional powder, the concentration of individual HMOs in the nutritional powder is preferably from about 0.001% to about 5%, including from about 0.01% to about 1% (by weight of the nutritional powder). Where the composition is a ready-to-feed nutritional liquid, the concentration of individual HMOs is preferably from about 0.001% to about 0.50%, including from about 0.001% to about 0.15%), including from about 0.01% to about 0.10%, and further including from about 0.01%) to about 0.03% (by weight of the ready-to-feed nutritional liquid). Where the composition is a concentrated nutritional liquid, the concentration of individual HMOs is preferably from about 0.002% to about 0.6%, including from about 0.002% to about 0.3%, including from about 0.02% to about 0.20% (by weight of the concentrated nutritional liquid).

The compositions of the present disclosure may optionally include anti-inflammatoirech as long-chain polyunsaturated fatty acids (LCPUFAs) and/or antioxidants such as carotenoids.

LCPUFAs may be included in the compositions to provide nutritional support and to enhance growth and functional development of the intestinal epithelium and associated immune cell populations. Exemplary LCPUFAs for use in the present compositions include, for example, docosahexaenoic acid (DHA), eicosapentaenoic acid (EPA), arachidonic acid (ARA), linoleic acid, linolenic acid (alpha linolenic acid) and gamma-linolenic acid derived from oil sources such as plant oils, marine plankton, fungal oils, and fish oils. The present compositions preferably comprise total concentrations of LCPUFA of from about 0.01 mM to about 10 mM and including from about 0.01 mM to about 1 mM. Alternatively, the compositions comprise total concentrations of LCPUFA of from about 0.001 g/L to about 1 g L.

Additionally, antioxidants such as carotenoids, and particularly, combinations of the carotenoids, lutein, lycopene, zeaxanthin and/or beta-carotene may be included in the present compositions. The compositions of the present disclosure may further comprise other optional components that may modify the physical, chemical, aesthetic or processing characteristics of the composition or to serve as pharmaceutical or additional nutritional components. Non-limiting examples of such optional ingredients include preservatives, emulsifying agents, buffers, pharmaceutical actives, nutrients, colorants, flavors, thickening agents and stabilizers, flowing agents, minerals, emulsifying agents, lubricants, sweetening agents, and the like.

A flowing agent or anti-caking agent may be included in the present compositions to retard clumping or caking of the powder over time and to make a powder embodiment flow easily from its container. Non-limiting examples include tricalcium phosphate, silicates, and combinations thereof. The concentration of the flowing agent or anti-caking agent in the nutritional composition varies depending upon the product form, the other selected ingredients, the desired flow properties, and so forth, but most typically range from about 0.1% to about 4%, including from about 0.5% to about 2%, by weight of the nutritional composition.

The compositions of the present disclosure may be prepared by any known or otherwise effective manufacturing technique for preparing the selected product solid or liquid form. Many such techniques are known for any given product form such as nutritional liquids or powders and can easily be applied by one of ordinary skill in the art to the nutritional compositions described herein.

Those skilled in the art will appreciate that the administration of compositions disclosed herein can be carried out with dose levels and dosing regimens as required depending on the circumstances and on the condition of the subject. Suitable dosage regimes can be determined based on the teaching of the present application. Dosage regimens may be adjusted to provide the optimal support of the subject. It will be appreciated that the exact amounts and rates of administration will depend on a number of factors such as the age, body weight, general health, sex, and dietary requirements of the subject. Based on the teaching herein those skilled in the art can, by routine trial and experimentation, determine suitable dosage regimes on a case-by-case basis.

The present compositions may comprise at least one probiotic strain, for example, Lactococcus lactis subsp. lactis biovar. diacetylactis, Lactococcus lactis subsp. cremoris, Lactococcus lactis subsp. lactis, any strain belonging to the genus Lactobacillus (including but not limited to Lactobacillus acidophilus, Lactobacillus casei subsp. casei, Lactobacillus delbrueckii subsp. bulgaricus, Lactobacillus fermentum, Lactobacillus gasseri, Lactobacillus helveticus, Lactobacillus lactis, Lactobacillus rhamnosus, Lactobacillus salivarius), any strain belonging to the genus Bifidobacterium (including but not limited to Bifidobacterium adolescentis, Bifidobacterium angulatum, Bifidobacterium animalis subsp. lactis, Bifidobacterium bifidum, Bifidobacterium breve, Bifidobacterium catenulatum, Bifidobacterium dentium, Bifidobacterium longum subsp. infantis, Bifidobacterium longum subsp. longum, Bifidobacterium magnum, Bifidobacterium pseudocatenulatum), or any strain from the genera of Akkermansia, Anaerostipes, Butyricicoccus, Christensenella, Clostridia, Coprococcus, Dorea, Eubacterium, Faecalibacterium or Roseburia or the family Coriobacteriaceae, as well as suitable combinations of the foregoing.

The present compositions may comprise at least one strain of a bacterium selected from the group comprising Bifidobacterium animalis subsp. lactis deposited as DSM 15954, Lactobacillus acidophilus deposited as DSM 13241, Lactobacillus rhamnosus deposited as ATCC 53103, Lactobacillus paracasei subsp. paracasei deposited as ATCC 55544, Lactobacillus paracasei deposited as LMG-17806, Streptococcus thermophilus deposited as DSM 15957, Lactobacillus fermentum deposited as NM02/31074, Lactobacillus paracasei subsp. paracasei deposited as CCTCC M204012 and suitable combinations thereof.

The present compositions preferably comprise an effective amount of probiotic. For example, where present it is preferred the probiotic has a concentration ranging from 0.05×10⁹ CFU/g to 30×10⁹ CFU/g, preferably from 0.5×10⁹ CFU/g to 25×10⁹ CFU/g.

### EXAMPLES

The 5 HMO-Mix containing: 2.99 mg/ml 2′-FL, 0.75 mg/ml 3-FL, 1.5 mg/ml LNT, 0.23 mg/ml, 3′-SL, and 0.28 mg/ml 6′-SL, was produced by Chr. Hansen HMO GmbH, Rheinbreitbach, Germany. Stocks 5-HMO solutions were dissolved in water.

Human monocytes were isolated from PBMC using Easysep Human Monocyte Enrichment Kit following manufacturer's protocol. To generate classical macrophages, 1.5×10⁶ of purified monocytes were cultured in flat-bottomed 6-well plates (Cat: Nunc) during 6 days in 3 ml FBS medium ((RPMI-1640 medium (Cat: R5886, Sigma Aldrich) with 1% streptomycin, 1% penicillin, 1% L-glutamine, and 10 % heat-inactivated and endotoxin-free fetal bovine serum (FBS) (Cat: 10082-147, Gibco) in the presence of GM-CSF (50 ng/ml, Cat: AF-HDC, Peprotech). To generate dendritic cells (DCs), 1.5×10⁶ of purified monocytes were cultured in flat-bottomed 6-well plates (Cat: Nunc) during 6 days in 3 ml FBS medium ((RPMI-1640 medium (Cat: R5886, Sigma Aldrich) with 1% streptomycin, 1% penicillin, 1% L-glutamine, and 10 % heat-inactivated and endotoxin-free fetal bovine serum (FBS) (Cat: 10082-147, Gibco) in the presence of GM-CSF and IL-4 (50 ng/ml, Cat: AF-HDC, Peprotech). After 3 days, FBS medium and cytokines were replenished. On day 5, differentiated DCs and MØ were washed with PBS and resuspended in X-VIVO 15 medium (Cat: Lonza). Subsequently, naïve MØ were stimulated with LPS (50 ng/ml) (Cat: 5568, Sigma-Aldrich) and IFNy (50 ng/ml) (Cat: 285-IF-100/CF, R&D Systems), and naïve DCs were activated with LPS (50 ng/ml) (Cat: 5568, Sigma-Aldrich) in the presence of 5-HMO Mix increasing concentrations (1-5 mg/ml) for 24 hours. For co-culture experiments, −+ LPS and 5-HMO Mix-stimulated MØ and DCs were collected, washed with PBS and resuspended in X-VIVO 15 medium. Subsequently, 1×10⁵ of MØ and DCs were co-cultured with 1×10⁶ naïve CD4⁺ T cells in flat-bottomed, 24-well culture plates (Cat: Nunc) for 5 days. Hereafter, supernatants were collected for cytokines analysis.

All procedures involving the handling of human samples were in accordance with the principles described in the Declaration of Helsinki and the samples were collected and analyzed according to ethically approval by the Regional Ethical Committee of the Capital Region of Denmark (H-16033682). Peripheral blood mononuclear cells (PBMCs) were isolated from healthy donors' blood by density gradient centrifugation using Lymphoprep (1114547, Axis-Shield, Oslo, Norway). Subsequently, naïve CD4⁺ T cells were purified by negative selection using Easysep Human Naive CD4⁺ T cell Enrichment Kit (Cat: 19155 Stemcell Technologies) according to manufacturer's protocol.

In brief, antibodies targeting undesired cells were supplemented to the PBMCs, and subsequently magnetic particles were used to bind desired cells. Next, these cells were retained using EasySep Magnet (Cat: 18000, Stemcell Technologies), and the resulting cell population consists of >95% naïve CD4⁺ T cells. The obtained naïve CD4⁺ T cells were counted by optical microscopy. For co-culture experiments, naïve CD4⁺ T cells were cultured with allogeneic macrophages either allogeneic DCs in a 1:10 ratio (MØ:TC) (DC:TC) in serum-free X-VIVO 15 medium (Cat: BE02-060F, Lonza, Verviers, Belgium) in flat-bottomed 24 well culture plates (Cat: 142475, Nunc) for 5 days.

To determine the effects of HMOs on cytokine secretion from mono-and co-cultures, the concentration of IL-4, IL-6, IL-10, IL-12 and IL-23 in mono-cultures (DCs or MØ), and IFN-γ, IL-13 and IL-17 in co-cultures (DC-CD4+ T-cells or MØ-CD4+ T cells) were measured by ELISA according to the manufacturer's instruction (InVitroGen, IFNG Cat: 88-7316-88 and IL-13 Cat: 88-7439-88). Following stimulation with LPS and 5-HMO Mix for 24 h, supernatants were isolated and stored at −80C. Subsequently the supernatants were analyzed for cytokines using the U-PLEX Panel Human Kit (K15049D), Meso Scale Diagnostics, Maryland, USA). Supernatants were incubated in plates with pre-coated wells with capture-antibodies. The plates were washed and incubated with capture antibodies wee against IL-10, IL-12p70, and IL-23 for 2 hours. The plates were washed again and incubated with secondary SULFO-TAG detection antibodies for 1 h. Finally, the plates were analyzed using the MESO QuickPlex SQ 120 and Discovery Workbench® v4.0.

High bind 96-well MaxiSorp Immunoplates (Thermo Scientific, Cat. 439454) were coated with 100 μl of PBS containing 2 μg/mL B. pertussis Toxin antigen (Creative Diagnostics, Cat. DAGH045) overnight at 4° C. in a humidified chamber. Coated plates were washed three times with 300 μl wash buffer ((1X PBS, 0/05% Tween-20 (Sigma, Cat. P1379)). Non-specific binding sites were blocked by incubating plates for 60 min at RT without shaking with blocking solution ((5% skim milk (BD, Cat.232100) in wash buffer). After blocking, plates were washed three times with 300 μl of wash buffer, then samples, standard and control diluted in blocking buffer were added in corresponding wells.

Standard and positive control are pool of mouse sera obtained from mice vaccinated with DTaP. The negative control used was naïve mouse serum. The standard curve (Nexelis, Lot. 09Nov.2022) starting dilution was 1/200. Serum samples starting dilution were either 1/50 or 1/1000. Positive (Nexelis, Lot. 09Nov.2022) and negative control serum starting dilution were 1/50 and 1/50, respectively. Samples, standard, and controls were serially diluted 2-fold in subsequent rows using pre-dispensed blocking buffer. Then plates were incubated for 1 hr at RT on an orbital shaker set at 350 rpm. Plates were washed 3 times with wash buffer and 100 μl of HRP-conjugated goat anti-mouse IgG (Bethyl, Cat. A90-116P) antibody 1/15000 diluted in blocking buffer was added. After 1 hr incubation at RT on an orbital plate shaker set at 350 rpm, plates were washed 5 times with 300 μl of wash buffer. One hundred ul of 3,3′,5,5′-Tetramethylbenzidine (TMB) substrate (Bio-Rad, Cat. 1721072) was added to all wells and plates were incubated for 30 minutes at RT protect from light. Enzymatic reaction was stopped by adding 100 μl of Stop Solution (Sulphuric acid, 0.36N), and optical density (OD) was read at 450 nm with a microplate reader connected to a network PC equipped with the SoftMax Pro software (version 7).

Standard curves were fit using 4-parameters logistic equation. Sample concentrations were determined for each dilution within the interpolation range. At least two concentration values were required per sample. The geometric mean concentration was used to report the final concentration. If the concentration value was close to the starting dilution of 1/50, then one value was accepted as samples cannot be tested at a lower dilution.

The EC50 values of standard curves generated during the anti-PT specific IgG ELISA development experiments were compiled. The geometric mean concentration of the EC50 values were calculated and an arbitrary concentration of 2704 ELU/ml was determined for the standard. The positive control was determined by obtaining the reference value ±2 SD.

Acceptance criteria for each assay plate:
Standard duplicate CV≤30%
Standard curve R2≥0.985
Sample CV≤30%, and n≥2 points, unless close to starting dilution
Sample interpolation: Samples with concentration <21 ELU/ml (corresponding to LLOQ) will be reported as <21 ELU/ml
The Positive control acceptance range was: 236-308 ELU/ml

Spleen cell suspensions were prepared by gently crushing spleens through 100 μm cell strainers placed on a tube containing 48 mL of plain RPMI-1640 (Lonza, Cat. 12-167Q) with plunger part of a 3-5 mL syringe piston. Cell suspensions were centrifuged and washed with plain RPMI. After transfer of cell suspension to 15 ml falcon tubes, cells were centrifuged at 400 g for 10 min at RT and cell pellet resuspend in 10 ml of complete RPMI (RPMI-1640 (Lonza, Cat. 12-167Q), containing 10% FBS (Gibco, Cat. 12483-020), 100 U/mL Penicillin- 100 μg/mL Streptomycin (Gibco, Cat. 15140-122), 1x MEM non-essential amino acids (Gibco, Cat. 11140-050), 1 mM sodium pyruvate (Gibco, Cat. 11360-070), 1x GlutaMAX (Gibco, Cat. 35050-061) and 5 μM β-mercaptoethanol (Gibco, Cat. 21985-023)) before being counted with Vi-Cell BLU (Beckmann Coulter Life Sciences) for flow cytometry assay.

Briefly, 1×10⁶ cells/well were stimulated with PBS, Pertussis Toxin (PT) (8 μg/mL) and PMA/IONO positive control (6.25 ng/ml for PMA and 0.125 μg/mL for IONO) at 37° C. under 5% CO2.

Brefeldin A (GolgiPlug (BD, Cat 51-2301KZ) and Monensin (GolgiStop BD, Cat 51-2092KZ), intracellular protein transport blocking agents, were added 2hrs after the initiation of the stimulation to inhibit cytokine transport and secretion. Following an overnight incubation (17 hr±1 hr) at 37° C. under 5% CO2, cells were harvested, washed with D-PBS and stained with Fixable Viability Stain 780. After 15 mins at RT protected from light, cells were washed with staining buffer and stained with surface markers (For panel 1: CD4, CD8, CD69 and for panel 2: CD4, CD8, CD62L, CD25, CD44). After 30 mins at RT, cells were washed with staining buffer and resuspended with: for panel 1: Cytofix-Cytoperm solution (BD, Cat. 554722) for 20 mins at 4° C. on ice, for panel 2 TF Perm/wash buffer (BD, 562574) for 45 mins at 4° C. on ice. Cells were then washed twice with 1X Perm/Wash buffer (for panel 1: BD, Cat. 554723, for Panel 2: BD, Cat. 562574). Cells were then stained with intracellular marker antibodies (for panel 1: CD3, IFN-γ, TNF-α, IL-2, IL-13 and for panel 2: CD3, KI-67, T-bet, Foxp3) 30 mins at RT. After intracellular staining, cells were washed with 1X Perm/Wash buffer, fixed with 1% PFA (EMS, Cat. 15735-20S) fixation solution and kept at 4° C. protected from light until acquisition on flow cytometer.

For identification of positive and negative populations, the fluorescence minus one (“FMO”) principle was used to account for background antibody fluorescence. Compensation was performed using single stained compensation beads. Acquisition of each well (170 μl) was performed as described below. All events were saved during acquisition.

Samples were acquired on a BD LSRFortessa™ X-20 (BD Biosciences), using BD FACSDiva™ v9.0 software (BD Biosciences). Analysis was performed using a validated FlowJo software v10.7.1.

### Effects of 5-HMO Mix Supplementation in Human Monocyte Derived Macrophages

We stimulated monocyte-derived MØ for 24 hours in the presence of LPS and 5-HMO Mix increasing concentrations, and measured the concentration of IL-10, IL-12 and IL-23 in the supernatants. We found that 5-HMO Mix supplementation dose-dependently increased the production of pro-inflammatory cytokines IL-12 and IL-23, IL-6 and TNF. Interestingly, we also found that 5-HMO Mix dose-dependently promoted IL-10 production.

### Immunomodulatory Effects of 5-HMO Mix on Dendritic Cells

We stimulated monocyte-derived DCs for 24 hours in the presence of LPS and 5-HMO Mix increasing concentrations, and measured the concentration of IL-10, IL-12, and IL-23in the supernatant. We found that 5-HMO Mix supplementation dose-dependently increased the production of IL-10, IL-12 and IL-23.

### Stimulated T Cells by Allogeneic 5-HMO Mix-conditioned MØ and DCs Support Th1 Responses Whilst Regulate Th2 Immunity

We co-cultured +LPS+5-HMO Mix-conditioned MØ and DCs with naïve CD4⁺ T cells. At day 5 we measured the concentration of IFN-γ and IL-13 in the supernatant. In co-cultures of MØ activating T cells, we found that 5-HMO Mix supplementation led to a dose-dependent significant increase in IFN-γ secretion. Conversely, 5-HMO Mix supplementation significantly decreased the production of IL-13 in allogeneic activated T cells with DCs. The IL-13/IFN-γ ratio in the T cell supernatant using the mean values for all donor pairs showed that +LPS+HMO-conditioned MØs enhanced Th1 and lowered Th2 response.

### Stimulated T Cells by Allogeneic 5-HMO Mix-conditioned DCs Support Th1 Responses Whilst Regulate Th2Immunity

To determine how 5-HMO Mix modulate T cell responses through influencing macrophage and dendritic cell function, we co-cultured +LPS+5-HMO Mix-conditioned MØ and DCs with naïve CD4⁺ T cells. At day 5 we measured the concentration of IFN-γ and IL-13 in the supernatant. In co-cultures of DC activating T cells, we found that 5-HMO Mix supplementation led to a dose-dependent significant increase in IFN-y secretion. Conversely, 5-HMO Mix supplementation significantly decreased the production of IL-13 in allogeneic activated T cells with DCs. The IL-13/IFN-γ ratio in the T cell supernatant using the mean values for all donor pairs showed that +LPS+HMO-conditioned MØs enhanced Th1 and lowered Th2 response.

1. A composition for use in balancing the immune system of a newborn or infant subject, said composition comprising an effective amount of 2′-fucosyllactose, 3-fucosyllactose, 3′-sialyllactose, 6′-sialyllactose, and lacto-N-tetraose.

2. The composition of claim 1, wherein the composition is a nutritional composition.

3. The composition of claim 2, wherein the composition is a powder.

4. The composition of claim 3, where the composition comprises at least 0.01%, by weight, of 2′-fucosyllactose; at least 0.01%, by weight, 3-fucosyllactose; at least 0.01%, by weight, 3′-sialyllactose; at least 0.01%, by weight, 6′-sialyllactose; and at least 0.01%, by weight, lacto-N-tetraose (LNT).

5. The composition of claim 3, wherein the 2′-fucosyllactose has particle size distribution, as measured using sieves complying to DIN ISO 3310-1, of less than about 20% through mesh #230 (63 μm), greater than about 65% through mesh #100 (150 μm), greater than about 92% through mesh #45 (355 μm), and 100% through mesh #20 (850 μm).

6. The composition of claim 1, wherein the composition comprises at least 0.5×10⁹ CFU/g of a probiotic.

7. The composition of claim 1, wherein the subject is a newborn.
